(19) United States
(12) Patent Application Publication
(10) Pub. No.: US 2026/0234518 A1
(43) Pub. Date: Aug. 13, 2026

(54) METHOD FOR PREPARING NURUK-FERMENTED CARBONATED BEVERAGE AND NURUK-FERMENTED CARBONATED BEVERAGE PREPARED THEREBY

(71) Applicant: Boksoondoga Co., Ltd., Ulsan (KR)

(72) Inventors: Minkyu Kim, Ulsan (KR); Minkook Kim, Ulsan (KR)

(73) Assignee: Boksoondoga Co., Ltd., Ulsan (KR)

(21) Appl. No.: 19/052,415

(22) Filed: Feb. 13, 2025

(51) Int. Cl. C12G 3/022; A23L 5/10; A23L 7/104; A23L 31/10; C12G 3/025; C12N 1/14

(52) U.S. Cl. CPC C12G 3/022; A23L 5/13; A23L 7/104; A23L 31/10; C12G 3/025; C12N 1/14

(57) ABSTRACT

A method for preparing nuruk-fermented carbonated beverage, includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; (g) filtering the secondary fermented mixture; (h-1) adding yeast to the filtered mixture and then subjecting the resulting mixture to a third fermentation in a sealed container; and (i) rapidly cooling the container.

### BACKGROUND

### 1. Field of the Invention

The present invention relates to a method for preparing nuruk-fermented carbonated beverage and nuruk-fermented carbonated beverage prepared thereby, and more particularly, to a method for preparing nuruk-fermented carbonated beverage with excellent preference such as taste and aroma, and excellent saccharogenic power, and nuruk-fermented carbonated beverage prepared thereby.

### 2. Discussion of Related Art

Fermentation is one of the reaction processes that generate energy and organic products, and it is a process in which microorganisms obtain energy by decomposing organic nutrients such as glucose in an anaerobic environment. In addition, the microorganisms involved in fermentation themselves enter the human body and have a beneficial effect known as probiotics, and have a special effect on health. In 2006, three out of the five world's healthiest foods selected by the American magazine Health were fermented foods, and as various physiological activities due to the fermentation process became known, fermented foods are recognized worldwide as health functional foods. Since long ago, microorganisms have had a close relationship with human life, directly or indirectly, depending on their characteristics, from fermented dairy products to various sauces, kimchi, fermented sausages, medicines, and livestock feed additives. Fermented foods have a stronger flavor than conventional foods, produce bacteriocin, a substance that inhibits the growth of microorganisms, and have antibacterial activity, and produce a large amount of lactic acid to inhibit the growth of spoilage bacteria in foods (Biosci Biotech Bioch 61: 1211-1212, 1997). In addition, previous studies have reported that consumption of yogurt, one of the fermented foods, reduced the mortality rate due to heart disease and type 2 diabetes (Brit J Nutr 113: S131-S135). Fermented foods can be largely classified according to the raw materials used, and even when the same raw materials are used, foods produced in different countries around the world often have different characteristics. Most of the early fermented foods occurred naturally in nature, but by identifying the causative bacteria of fermentation, we have entered the stage of isolating excellent bacteria and artificially managing the entire fermentation process. Meanwhile, nuruk refers to the yeast mold that grows on cooked grains in a warm and humid environment, and specifically, under suitable conditions, fine yeast mold spores settle on cooked grains such as barley, wheat, and rice, and the germinated mycelia form a network known as mycelia, forming a white, fluffy mass of mold grains. Aspergillus oryzae is a fungus belonging to the Ascomycetes genus. It grows by taking in nutrients from various substances, many of which are used in industry. When it reproduces sexually, elongated saccules and saccules grow next to each other from multicellular mycelia, and these intertwine and fuse together. Afterwards, surrounding mycelia surround these, forming small, yellow fruiting bodies in the shape of small balls. The saccules that branch out from here grow irregularly to form asci, so the asci are dispersed and formed within the fruiting bodies. When the fruiting body matures, the perithecium wall melts and disappears, and the asci are released inside the fruiting body, and the asci are dispersed when the surrounding mycelial tissue collapses. On the other hand, in asexual reproduction, a conidial spore stalk is formed on the mycelium, and conidia are created at the end of the spore stalk. The general propagation method of the Aspergillus oryzae is this asexual reproduction.

The specific method of making Aspergillus oryzae is shown in the literature of the Joseon Dynasty. According to this, the materials used for Aspergillus oryzae were wheat, rice, mung beans, and barley. Later, it was divided into Bunguk made with wheat flour and Joguk made with wheat and wheat bran, and their uses were different. In some regions, oats, hulled barley, and barley mixed with sake lees were steamed and used as raw materials.

The production of Aspergillus oryzae was mostly done on a small scale in the summer and fall as a side job for farmers, but starting in 1927, Aspergillus oryzae manufacturers and brewers came together to establish a ladle manufacturing company, solidifying its position as a production industry. As a result, production that had previously been done only in the right season was now done all year round, and the quality improved and the products became uniform. After that, in regions where soju was produced, as nuruk was changed to black yeast for soju production, the production of nuruk gradually decreased, and only yakju and takju remained. In the 1940s, it was unified under the improved imperial law, and since 1950, various attempts have been made to improve nuruk. Depending on the material, nuruk can be made from wheat flour, nuruk made from rice and mung beans, nuruk made from autumn barley, and nuruk made only from rice flour. Depending on the manufacturing period, it was called chun-guk, ha-guk, jeol-guk, and dong-guk, but jeol-guk made after wheat harvest was the most common. Depending on the form, it can be divided into byeong-guk made by grinding grains and then lumping them together, and san-guk made from grains or flour, but byeong-guk was the most commonly used. Byeongguk was often made by directly using powder, but sometimes it is steeped in water or juice. Nowadays, it is divided into bunguk made from wheat flour and joguk made from wheat flour and bran, and their uses are different. Bunguk is used for yakju or gwahaju, and joguk is used for takju and soju.

The shape and manufacturing method vary depending on the region. In some regions, the raw materials are kneaded, wrapped in cloth, put into a mold, trampled tightly, wrapped in straw, and then deposited on the ondol. In other regions, instead of depositing, it is hung indoors. The shape also varies, such as spherical, conical, and hat-shaped.

The quality of nuruk is affected not only by the type of raw material used, but also by its shape. If the diameter of the nuruk is too short, moisture is easily released and bacteria do not penetrate well, resulting in poor maturation. If it is too thin, it matures in a short period of time, but the flavor is not good and the alcohol content is low. If it is too thick, the moisture inside is difficult to evaporate, which may increase the internal temperature, and it is also difficult to dry after manufacturing. The quality of nuruk varies depending on the degree of stepping, and the quality of nuruk is directly related to the taste of alcohol.

Nuruk has an excellent ability to break down the molecular structure of starch and protein into smaller pieces, allowing the nutrients, sweetness, and savory flavors of grains to be perfectly consumed and absorbed. Nuruk is used as a fermentation agent for making traditional liquors such as Korean makgeolli and yakju. Nuruk is made by crushing grains such as barley or wheat, adding moisture, forming them hard, and then exposing them to the environment to allow various fermentation microorganisms to grow. For this reason, the diversity and content of microorganisms in nuruk are determined by the geographical and physicochemical environment, such as the manufacturing region, temperature, and composition of raw materials.

However, since nuruk is fermented by naturally attached microorganisms, there are many opportunities for bacterial contamination, and the quality is unstable, making process management difficult. Meanwhile, Makgeolli is a traditional Korean alcoholic beverage made by mixing rice, water, and yeast, fermenting, filtering the fermented mash, and then adding water to lower the alcohol content to make it easier to drink. The word Makgeolli means a heavily filtered alcoholic beverage. This filtered alcoholic beverage is called Takju because it has a cloudy and cloudy color, Makgeolli because it is a very filtered alcoholic beverage, Gaju because it is a beverage made in every home, Baekju because it is a white alcoholic beverage, Nongju because it is a beverage consumed during farming, Jeju because it is served on the ancestral rite table, Hyangju because it is the most popular alcoholic beverage among the people on special occasions, and Gukju because it is Korea's representative alcoholic beverage. It is also called by various other names in each region.

Looking at the current state of the alcoholic beverage industry, there is a trend of preferring alcoholic beverages with low alcohol content, and the trend of health food is showing a very popular growth, but general distilled alcoholic beverages and distilled soju with high alcohol content are on the decline. In the case of makgeolli consumption, consumption of raw makgeolli is increasing significantly compared to pasteurized makgeolli, but since the distribution period is short, liquor companies that do not have refrigeration facilities require high initial capital.

Accordingly, the inventors of the present invention discovered specific process conditions that can overcome the limitations of nuruk and developed a method for manufacturing nuruk for fermentation, thereby completing the present invention. In addition, using this nuruk for fermentation, they developed a nuruk fermented beverage and a nuruk fermented carbonated beverage with excellent preferences in terms of taste and aroma, and developed a nuruk fermented cosmetic composition with excellent soothing, moisturizing, and wrinkle-improving effects.

### PATENT DOCUMENTS

Korean Patent Application Publication No. 10-2017-0098429

### SUMMARY OF THE INVENTION

Therefore, a problem to be solved by the present invention is to provide a method for preparing a nuruk-fermented carbonated beverage with excellent preference such as taste and aroma.

Yet another problem to be solved by the present invention is to provide a nuruk-fermented carbonated beverage prepared by the method.

Yet another problem to be solved by the present invention is to provide a method for preparing nuruk for fermentation, which can be used to prepare a nuruk-fermented beverage with excellent preference such as taste and aroma, and has excellent saccharogenic power.

Another problem to be solved by the present invention is to provide nuruk for fermentation prepared by the method.

Still another problem to be solved by the present invention is to provide a method for preparing a nuruk-fermented beverage using nuruk for fermentation prepared by the method, and the nuruk-fermented beverage may have excellent preference such as taste and aroma.

Yet another problem to be solved by the present invention is to provide a nuruk-fermented beverage prepared using nuruk for fermentation prepared by the method, and the nuruk-fermented beverage may have excellent preference such as taste and aroma.

Yet another problem to be solved by the present invention is to provide a method for preparing a nuruk-fermented carbonated beverage, and the nuruk-fermented carbonated beverage may have excellent preference such as taste and aroma.

Yet another problem to be solved by the present invention is to provide a nuruk-fermented carbonated beverage prepared using nuruk for fermentation prepared by the method, and the nuruk-fermented carbonated beverage may have excellent preference such as taste and aroma.

Yet another problem to be solved by the present invention is to provide a method for preparing a nuruk-fermented cosmetic composition using nuruk for fermentation prepared by the method, and the nuruk-fermented cosmetic composition may have excellent soothing, moisturizing, and anti-wrinkle effects.

Yet another problem to be solved by the present invention is to provide a nuruk-fermented cosmetic composition using nuruk for fermentation prepared by the method, and the nuruk-fermented cosmetic composition may have excellent soothing, moisturizing, and anti-wrinkle effects.

Yet another problem to be solved by the present invention is to provide a method for preparing a nuruk-fermented beverage with excellent preference such as taste and aroma.

Yet another problem to be solved by the present invention is to provide a nuruk-fermented beverage prepared by the method.

Yet another problem to be solved by the present invention is to provide a method for preparing a nuruk-fermented cosmetic composition having excellent soothing, moisturizing, and anti-wrinkle effects.

Yet another problem to be solved by the present invention is to provide a nuruk-fermented cosmetic composition prepared by the method.

The problems of the present invention are not limited to the aforementioned technical problems, and other technical problems, which have not been mentioned, may be clearly understood by a person with ordinary skill in the art from the following description.

A method for preparing nuruk-fermented carbonated beverage according to an embodiment of the present invention for solving the problems includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; (g) filtering the secondary fermented mixture; (h-1) adding yeast to the filtered mixture and then subjecting the resulting mixture to a third fermentation in a sealed container; and (i) rapidly cooling the container.

The nuruk may be obtained by a method comprising: (a) kneading a mixture of wheat and water; (b) leaving the resulting dough to stand for a predetermined time; (c) molding the dough into a predetermined shape; (d) a primary culture step of culturing the molded dough at a first fermenting temperature; (e) a secondary culture step of culturing the primary cultured dough at a second fermenting temperature lower than the first fermenting temperature; (f) drying the secondary cultured dough by lowering a fermenting temperature of the secondary cultured dough; and (g) aging the dried dough for a predetermined time.

The wheat in step (a) may include 70 to 90 parts by weight of wheat unhulled grains and 10 to 30 parts by weight of wheat flour grains.

In step (a), nuruk prepared by the same method may be further mixed in an amount of 2 to 4 parts by weight with respect to 100 parts by weight of the wheat.

In step (d), one or more of pine needle and straw may be laid under or around the molded dough, and then the dough is cultured.

The first fermenting temperature may be 35 to 50° C. and the primary culture period may be 8 to 12 days.

The second fermenting temperature may be 30 to 45° C. and the secondary culture period may be 5 to 10 days.

The method may further comprise sprinkling wheat flour grains on the dough in an amount of 3 to 5 parts by weight with respect to 100 parts by weight of the wheat during the secondary culture in step (e).

The method may further comprise (c-1) inoculating the molded dough with a strain composition between steps (c) and (d).

The strain composition may comprise one or more selected from the group consisting of Aspergillus oryzae, Lichtheimia corymbifera, Monascus pilosus, Galactomyces candidus, and Rhizopus japonicus.

The strain composition may comprise Galactomyces candidus.

The strain composition may comprise Lichtheimia corymbifera, Monascus pilosus, and Galactomyces candidus.

The strain composition may comprise Lichtheimia corymbifera, Monascus pilosus, and Galactomyces candidus at a weight ratio of 2:1 to 2:1 to 2.

Each strain comprised in the strain composition may be obtained by a method comprising:
inoculating a culture medium with a specific strain;
culturing an inoculum at 20 to 25° C. for 48 hours;
inoculating a new culture medium with the culture solution again to perform culture at 20 to 25° C. for 48 hours; and
collecting a culture product by centrifugation.

The strain composition may further comprise honey.

The step (d) may be a step of mixing 80 to 120 parts by weight of the cooled raw material, 5 to 20 parts by weight of nuruk, and 50 to 150 parts by weight of water.

The second temperature may be higher than the first temperature.

The first temperature in step (e) may be 20 to 30° C., and the primary fermentation period may be 1 to 4 weeks.

The second temperature in step (f) may be 25 to 35° C., and the secondary fermentation period may be 2 to 9 days.

The yeast may be beer yeast.

A method for preparing nuruk-fermented carbonated beverage according to the other embodiment of the present invention for solving the problems includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; (g) filtering the secondary fermented mixture; and (h-2) injecting carbon dioxide gas into the filtered mixture and then sealing the mixture.

The nuruk may be obtained by a method comprising: (a) kneading a mixture of wheat and water; (b) leaving the resulting dough to stand for a predetermined time; (c) molding the dough into a predetermined shape; (d) a primary culture step of culturing the molded dough at a first fermenting temperature; (e) a secondary culture step of culturing the primary cultured dough at a second fermenting temperature lower than the first fermenting temperature; (f) drying the secondary cultured dough by lowering a fermenting temperature of the secondary cultured dough; and (g) aging the dried dough for a predetermined time.

The wheat in step (a) may include 70 to 90 parts by weight of wheat unhulled grains and 10 to 30 parts by weight of wheat flour grains.

In step (a), nuruk prepared by the same method may be further mixed in an amount of 2 to 4 parts by weight with respect to 100 parts by weight of the wheat.

In step (d), one or more of pine needle and straw may be laid under or around the molded dough, and then the dough is cultured.

The first fermenting temperature may be 35 to 50° C. and the primary culture period may be 8 to 12 days.

The second fermenting temperature may be 30 to 45° C. and the secondary culture period may be 5 to 10 days.

The method may further comprise sprinkling wheat flour grains on the dough in an amount of 3 to 5 parts by weight with respect to 100 parts by weight of the wheat during the secondary culture in step (e).

The method may further comprise (c-1) inoculating the molded dough with a strain composition between steps (c) and (d).

The strain composition may comprise one or more selected from the group consisting of Aspergillus oryzae, Lichtheimia corymbifera, Monascus pilosus, Galactomyces candidus, and Rhizopus japonicus.

The strain composition may comprise Galactomyces candidus.

The strain composition may comprise Lichtheimia corymbifera, Monascus pilosus, and Galactomyces candidus.

The strain composition may comprise Lichtheimia corymbifera, Monascus pilosus, and Galactomyces candidus at a weight ratio of 2:1 to 2:1 to 2.

Each strain comprised in the strain composition may be obtained by a method comprising:
inoculating a culture medium with a specific strain;
culturing an inoculum at 20 to 25° C. for 48 hours;
inoculating a new culture medium with the culture solution again to perform culture at 20 to 25° C. for 48 hours; and
collecting a culture product by centrifugation.

The strain composition may further comprise honey.

The step (d) may be a step of mixing 80 to 120 parts by weight of the cooled raw material, 5 to 20 parts by weight of nuruk, and 50 to 150 parts by weight of water.

The second temperature may be higher than the first temperature.

The first temperature in step (e) may be 20 to 30° C., and the primary fermentation period may be 1 to 4 weeks.

The second temperature in step (f) may be 25 to 35° C., and the secondary fermentation period may be 2 to 9 days.

A method for preparing nuruk-fermented carbonated beverage according to the other embodiment of the present invention for solving the problems includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; (g) filtering the secondary fermented mixture; (h-3) injecting yeast and carbon dioxide gas into the filtered mixture and then subjecting the resulting mixture to a third fermentation in a sealed container; and (i) rapidly cooling the container.

The nuruk may be obtained by a method comprising: (a) kneading a mixture of wheat and water; (b) leaving the resulting dough to stand for a predetermined time; (c) molding the dough into a predetermined shape; (d) a primary culture step of culturing the molded dough at a first fermenting temperature; (e) a secondary culture step of culturing the primary cultured dough at a second fermenting temperature lower than the first fermenting temperature; (f) drying the secondary cultured dough by lowering a fermenting temperature of the secondary cultured dough; and (g) aging the dried dough for a predetermined time.

The wheat in step (a) may include 70 to 90 parts by weight of wheat unhulled grains and 10 to 30 parts by weight of wheat flour grains.

In step (a), nuruk prepared by the same method may be further mixed in an amount of 2 to 4 parts by weight with respect to 100 parts by weight of the wheat.

In step (d), one or more of pine needle and straw may be laid under or around the molded dough, and then the dough is cultured.

The first fermenting temperature may be 35 to 50° C. and the primary culture period may be 8 to 12 days.

The second fermenting temperature may be 30 to 45° C. and the secondary culture period may be 5 to 10 days.

The method may further comprise sprinkling wheat flour grains on the dough in an amount of 3 to 5 parts by weight with respect to 100 parts by weight of the wheat during the secondary culture in step (e).

The method may further comprise (c-1) inoculating the molded dough with a strain composition between steps (c) and (d).

The strain composition may comprise one or more selected from the group consisting of Aspergillus oryzae, Lichtheimia corymbifera, Monascus pilosus, Galactomyces candidus, and Rhizopus japonicus.

The strain composition may comprise Galactomyces candidus.

The strain composition may comprise Lichtheimia corymbifera, Monascus pilosus, and Galactomyces candidus.

The strain composition may comprise Lichtheimia corymbifera, Monascus pilosus, and Galactomyces candidus at a weight ratio of 2:1 to 2:1 to 2.

Each strain comprised in the strain composition may be obtained by a method comprising:
inoculating a culture medium with a specific strain;
culturing an inoculum at 20 to 25° C. for 48 hours;
inoculating a new culture medium with the culture solution again to perform culture at 20 to 25° C. for 48 hours; and
collecting a culture product by centrifugation.

The strain composition may further comprise honey.

The step (d) may be a step of mixing 80 to 120 parts by weight of the cooled raw material, 5 to 20 parts by weight of nuruk, and 50 to 150 parts by weight of water.

The second temperature may be higher than the first temperature.

The first temperature in step (e) may be 20 to 30° C., and the primary fermentation period may be 1 to 4 weeks.

The second temperature in step (f) may be 25 to 35° C., and the secondary fermentation period may be 2 to 9 days.

The yeast may be beer yeast.

A method for preparing nuruk according to an embodiment of the present invention for solving the problems includes: (a) kneading a mixture of wheat and water; (b) leaving the resulting dough to stand for a predetermined time; (c) molding the dough into a predetermined shape; (d) a primary culture step of culturing the molded dough at a first fermenting temperature; (e) a secondary culture step of culturing the primary cultured dough at a second fermenting temperature lower than the first fermenting temperature; (f) drying the secondary cultured dough by lowering a fermenting temperature of the secondary cultured dough; and (g) aging the dried dough for a predetermined time.

The wheat in step (a) may include 70 to 90 parts by weight of wheat unhulled grains and 10 to 30 parts by weight of wheat flour grains.

In step (a), nuruk prepared by the same method may be further mixed in an amount of 2 to 4 parts by weight with respect to 100 parts by weight of the wheat.

In step (d), one or more of pine needle and straw may be laid under or around the molded dough, and then the dough is cultured.

The first fermenting temperature may be 35 to 50° C. and the primary culture period may be 8 to 12 days.

The second fermenting temperature may be 30 to 45° C. and the secondary culture period may be 5 to 10 days.

The method may further comprise sprinkling wheat flour grains on the dough in an amount of 3 to 5 parts by weight with respect to 100 parts by weight of the wheat during the secondary culture in step (e).

The method may further comprise (c-1) inoculating the molded dough with a strain composition between steps (c) and (d).

The strain composition may comprise one or more selected from the group consisting of Aspergillus oryzae, Lichtheimia corymbifera, Monascus pilosus, Galactomyces candidus, and Rhizopus japonicus.

The strain composition may comprise Galactomyces candidus.

The strain composition may comprise Lichtheimia corymbifera, Monascus pilosus, and Galactomyces candidus.

The strain composition may comprise Lichtheimia corymbifera, Monascus pilosus, and Galactomyces candidus at a weight ratio of 2:1 to 2:1 to 2.

Each strain comprised in the strain composition may be obtained by a method comprising:
inoculating a culture medium with a specific strain;
culturing an inoculum at 20 to 25° C. for 48 hours;
inoculating a new culture medium with the culture solution again to perform culture at 20 to 25° C. for 48 hours; and
collecting a culture product by centrifugation.

The strain composition may further comprise honey.

A method for preparing nuruk-fermented beverage according to an embodiment of the present invention for solving the problems includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; and (g) filtering the secondary fermented mixture.

The nuruk may be obtained by a method comprising: (a) kneading a mixture of wheat and water; (b) leaving the resulting dough to stand for a predetermined time; (c) molding the dough into a predetermined shape; (d) a primary culture step of culturing the molded dough at a first fermenting temperature; (e) a secondary culture step of culturing the primary cultured dough at a second fermenting temperature lower than the first fermenting temperature; (f) drying the secondary cultured dough by lowering a fermenting temperature of the secondary cultured dough; and (g) aging the dried dough for a predetermined time.

The wheat in step (a) may include 70 to 90 parts by weight of wheat unhulled grains and 10 to 30 parts by weight of wheat flour grains.

In step (a), nuruk prepared by the same method may be further mixed in an amount of 2 to 4 parts by weight with respect to 100 parts by weight of the wheat.

In step (d), one or more of pine needle and straw may be laid under or around the molded dough, and then the dough is cultured.

The first fermenting temperature may be 35 to 50° C. and the primary culture period may be 8 to 12 days.

The second fermenting temperature may be 30 to 45° C. and the secondary culture period may be 5 to 10 days.

The method may further comprise sprinkling wheat flour grains on the dough in an amount of 3 to 5 parts by weight with respect to 100 parts by weight of the wheat during the secondary culture in step (e).

The method may further comprise (c-1) inoculating the molded dough with a strain composition between steps (c) and (d).

The strain composition may comprise one or more selected from the group consisting of Aspergillus oryzae, Lichtheimia corymbifera, Monascus pilosus, Galactomyces candidus, and Rhizopus japonicus.

The strain composition may comprise Galactomyces candidus.

The strain composition may comprise Lichtheimia corymbifera, Monascus pilosus, and Galactomyces candidus.

The strain composition may comprise Lichtheimia corymbifera, Monascus pilosus, and Galactomyces candidus at a weight ratio of 2:1 to 2:1 to 2.

Each strain comprised in the strain composition may be obtained by a method comprising:
inoculating a culture medium with a specific strain;
culturing an inoculum at 20 to 25° C. for 48 hours;
inoculating a new culture medium with the culture solution again to perform culture at 20 to 25° C. for 48 hours; and
collecting a culture product by centrifugation.

The strain composition may further comprise honey.

The step (d) may be a step of mixing 80 to 120 parts by weight of the cooled raw material, 5 to 20 parts by weight of nuruk, and 50 to 150 parts by weight of water.

The second temperature may be higher than the first temperature.

The first temperature in step (e) may be 20 to 30° C., and the primary fermentation period may be 1 to 4 weeks.

The second temperature in step (f) may be 25 to 35° C., and the secondary fermentation period may be 2 to 9 days.

A nuruk-fermented cosmetic composition according to an embodiment of the present invention for solving the problems comprises a nuruk-fermented product, wherein the nuruk-fermented product is a fermented product of nuruk obtained by a method comprising: (a) kneading a mixture of wheat and water; (b) leaving the resulting dough to stand for a predetermined time; (c) molding the dough into a predetermined shape; (d) a primary culture step of culturing the molded dough at a first fermenting temperature; (e) a secondary culture step of culturing the primary cultured dough at a second fermenting temperature lower than the first fermenting temperature; (f) drying the secondary cultured dough by lowering a fermenting temperature of the secondary cultured dough; and (g) aging the dried dough for a predetermined time.

The wheat in step (a) may include 70 to 90 parts by weight of wheat unhulled grains and 10 to 30 parts by weight of wheat flour grains.

In step (a), nuruk prepared by the same method may be further mixed in an amount of 2 to 4 parts by weight with respect to 100 parts by weight of the wheat.

In step (d), one or more of pine needle and straw may be laid under or around the molded dough, and then the dough is cultured.

The first fermenting temperature may be 35 to 50° C. and the primary culture period may be 8 to 12 days.

The second fermenting temperature may be 30 to 45° C. and the secondary culture period may be 5 to 10 days.

The method may further comprise sprinkling wheat flour grains on the dough in an amount of 3 to 5 parts by weight with respect to 100 parts by weight of the wheat during the secondary culture in step (e).

The method may further comprise (c-1) inoculating the molded dough with a strain composition between steps (c) and (d).

The strain composition may comprise one or more selected from the group consisting of Aspergillus oryzae, Lichtheimia corymbifera, Monascus pilosus, Galactomyces candidus, and Rhizopus japonicus.

The strain composition may comprise Galactomyces candidus.

The strain composition may comprise Lichtheimia corymbifera, Monascus pilosus, and Galactomyces candidus.

The strain composition may comprise Lichtheimia corymbifera, Monascus pilosus, and Galactomyces candidus at a weight ratio of 2:1 to 2:1 to 2.

Each strain comprised in the strain composition may be obtained by a method comprising:
inoculating a culture medium with a specific strain;
culturing an inoculum at 20 to 25° C. for 48 hours;
inoculating a new culture medium with the culture solution again to perform culture at 20 to 25° C. for 48 hours; and
collecting a culture product by centrifugation.

The strain composition may further comprise honey.

The nuruk-fermented product may be obtained by suspending nuruk in water, then filtering the suspension with a filter, concentrating the filtered suspension under reduced pressure, and then filtering the concentrate with a filter again.

The water may be an amount of 3- to 10-fold the weight of the nuruk.

Concentration under reduced pressure may be performed until the Brix is 5 to 10.

The size of the filter may be 1 to 10 m.

The nuruk-fermented product may include a fermented product of Galactomyces candidus.

Specific details of other embodiments are included in the detailed description.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a flow chart illustrating a method for preparing a nuruk-fermented carbonated beverage according to an embodiment of the present invention; and

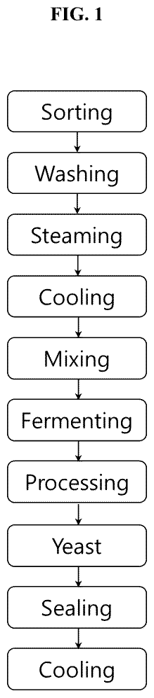

FIG. 2 is a flow chart illustrating a method for preparing a nuruk-fermented carbonated beverage according to another embodiment of the present invention.

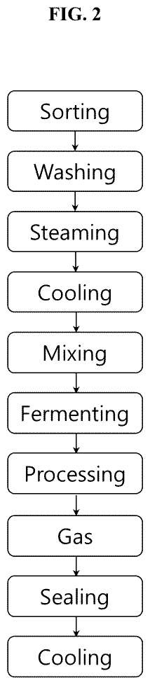

FIG. 3 is a flow chart illustrating a method for preparing nuruk according to an embodiment of the present invention;

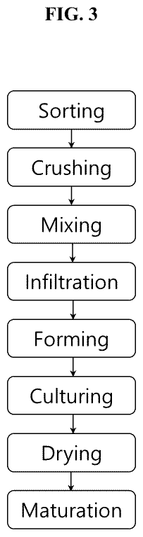

FIG. 4 is a photograph of nuruk prepared by the method according to an embodiment of the present invention;

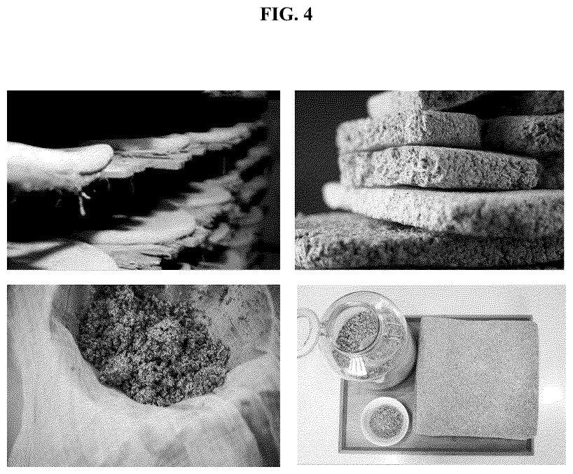

FIG. 5 is a flow chart illustrating a method for preparing a nuruk-fermented beverage according to an embodiment of the present invention;

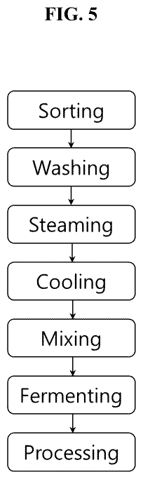

### DETAILED DESCRIPTION OF EXEMPLARY EMBODIMENTS

The advantages and features of the present invention, and the method for achieving them, will become clear with reference to the embodiments described in detail below. However, the present invention is not limited to the embodiments disclosed below, but may be implemented in various different forms, and the embodiments are provided only to make the disclosure of the present invention complete and to fully inform those skilled in the art of the invention of the scope of the invention, and the present invention is defined only by the scope of the claims. The terminology used in this specification is for the purpose of describing the embodiments and is not intended to limit the present invention. In this specification, ‘and/or’ includes each and every combination of one or more of the mentioned items. In addition, the singular also includes the plural unless specifically stated in the phrase. ‘comprises’ and/or ‘comprising’ as used in the specification do not exclude the presence or addition of one or more other components in addition to the mentioned components. The numerical range indicated using ‘-’ or ‘to’ indicates the numerical range including the values described before and after it as the lower and upper limits, respectively, unless otherwise stated. ‘About’ or ‘approximately’ means a value or numerical range within 20% of the value or numerical range described thereafter.

In addition, when describing the components of the embodiments of the present invention, terms such as first, second, A, B, (a), (b), etc. may be used. These terms are only intended to distinguish the components from other components, and the nature, order, or sequence of the components are not limited by the terms.

Unless otherwise defined, all terms (including technical and scientific terms) used in this specification may be used with a meaning that can be commonly understood by a person having ordinary knowledge in the technical field to which the present invention belongs. In addition, terms defined in commonly used dictionaries shall not be interpreted ideally or excessively unless explicitly specifically defined.

In addition, when describing the embodiments of the present invention, if a specific description of a related known configuration or function is judged to interfere with the understanding of the embodiments of the present invention, the detailed description will be omitted.

As used herein, “nuruk” refers to a fermenting agent made by propagating molds having alcohol-producing enzymes on grains, and may be classified into traditional nuruk, which is made by propagating molds, yeasts, bacteria, and the like, which are present in nature, and improved nuruk, which is made by inoculating sterilized starch raw materials with cultured bacteria, depending on the preparation method. The nuruk may be used in a broad sense to include all of yeast, leaven, malt, malted rice, and malted wheat.

As used herein, “fermentation” refers to the decomposition of organic matter occurring in anaerobic conditions where there is a lack of oxygen, and the fermentation method of the present invention includes all processes of fermentation using the nuruk prepared by the method of the present invention.

As used herein, “fermented food” refers to a food including the nuruk prepared by the method of the present invention, and may refer to a food that is fermented using the nuruk prepared by the method of the present invention, and may be prepared by a method typically used in the art. Specifically, the fermented food may be a fermented beverage, fermented salt, salted fish, fermented wine, vinegar, or soy sauce.

As used herein, “fermented beverage” refers to a beverage made by fermentation with the nuruk of the present invention, and specifically may include fermented wine, fermented tea, fermented oil, fermented soup, and the like, but includes any food without limitation, as long as it is fermented with the nuruk and can be consumed.

As used herein, “fermented carbonated beverage” refers to a carbonated beverage made by fermentation with the nuruk of the present invention, and includes any beverage without limitation, as long as it is fermented with the nuruk and includes carbon dioxide.

As used herein, “fermented wine” refers to wine obtained by mixing grains with the nuruk of the present invention and fermenting the mixture, and it is obvious in the art that the characteristics and taste of fermented wine vary depending on the raw material and nuruk. Makgeolli prepared using the nuruk of the present invention helps improve intestinal function because it is rich in lactic acid bacteria, and may exhibit antioxidant activity because it is rich in bioactive substances. For example, the fermented wine includes makgeolli, dong dong-ju, soju, seishu (refined rice wine), or the like.

As used herein, “fermented cosmetic composition” refers to a cosmetic composition including the nuruk, nuruk-fermented product, or nuruk extract of the present invention as an active ingredient, and includes, without limitation, its formulation, such as lotion, cream, toner, essence, and mask pack.

The “nuruk-fermented product” and “nuruk extract” of the present invention are fermented products or extracts prepared using nuruk itself, and include natural substances useful for beautiful skin, such as starch and minerals, which are contained in the raw material of nuruk, as well as enzymes and natural microorganisms including yeast, contained in large amounts in nuruk, and fermented products fermented by these microorganisms, and thus, may exhibit excellent efficacy in improving the skin.

The “fermented cosmetic composition” of the present invention may have excellent efficacy in improving skin condition through collagen synthesis effect, antioxidant effect through free radical scavenging activity, increasing skin moisturization by increasing aquaporin 3 (AQP3) and filaggrin (FLG) which retain moisture in the skin, and suppressing the production of nitric oxide which causes skin aging. In addition, since the fermented cosmetic composition includes natural substances as active ingredients, it has excellent effects such as anti-aging, skin soothing, skin barrier strengthening, and anti-wrinkle without problems associated with resistance or safety, and thus is useful for improving skin conditions, and may be used as a cosmetic composition for one or more uses of ameliorating skin wrinkles and improving skin elasticity, soothing the skin, strengthening the skin barrier, and skin antioxidant properties.

A method for preparing a nuruk-fermented carbonated beverage according to an embodiment of the present invention includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; (g) filtering the secondary fermented mixture; (h-1) adding yeast to the filtered mixture and then subjecting the resulting mixture to a third fermentation in a sealed container; and (i) rapidly cooling the container.

The yeast may be beer yeast, and specifically, it may be obtained by drying the residue after filtration of beer. The yeast may be added in an amount of 0.001 to 0.09 parts by weight with respect to 100 parts by weight of the filtered mixture.

The sealed container may be one that prevents the leakage of carbon dioxide gas generated during the tertiary fermentation process.

The tertiary fermentation may be performed at 10 to 20° C. for 10 to 20 hours. Alternatively, the tertiary fermentation may be performed until the pressure of the carbon dioxide gas in the sealed container reaches a predetermined pressure, specifically, 0.5 to 1.5 kg/cm².

The rapidly cooling of the container may be a step of first immersing the container in hot water at 50 to 55° C. for 10 to 20 minutes, and then rapidly cooling the container to 0 to 5° C.

The detailed description of the other steps (a) to (g) will be omitted because it has been described below.

A method for preparing a nuruk-fermented carbonated beverage according to another embodiment of the present invention includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; (g) filtering the secondary fermented mixture; and (h-2) injecting carbon dioxide gas into the filtered mixture and then sealing the mixture.

The carbon dioxide gas may be injected in an amount of 0.05 to 1 part by weight with respect to 100 parts by weight of the filtered mixture.

The detailed description of the other steps (a) to (g) will be omitted because it has been described below.

A method for preparing a nuruk-fermented carbonated beverage according to another embodiment of the present invention includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; (g) filtering the secondary fermented mixture; (h-3) injecting yeast and carbon dioxide gas into the filtered mixture and then subjecting the resulting mixture to a third fermentation in a sealed container; and (i) rapidly cooling the container.

The detailed description of steps (a) to (i) will be omitted because it has been described below.

A method for preparing nuruk according to an embodiment of the present invention includes (a) kneading a mixture of wheat and water; (b) leaving the resulting dough to stand for a predetermined time; (c) molding the dough into a predetermined shape; (d) a primary culture step of culturing the molded dough at a first fermenting temperature; (e) a secondary culture step of culturing the primary cultured dough at a second fermenting temperature lower than the first fermenting temperature; (f) drying the secondary cultured dough by lowering a fermenting temperature of the secondary cultured dough; and (g) aging the dried dough for a predetermined time.

In step (a), the amount of wheat used may be 100 to 150 parts by weight, specifically 110 to 130 parts by weight, and the amount of water used may be 10 to 50 parts by weight, specifically 20 to 40 parts by weight.

The wheat used in step (a) may include 70 to 90 parts by weight of wheat unhulled grains and 10 to 30 parts by weight of wheat flour grains. The wheat unhulled grains or flour grains may be prepared by a method including separating wheat kernels and stones in a winnowing machine and grinding the wheat kernels into unhulled grains or flour grains. In this case, the wheat kernels may be ground coarsely such that no raw grains remain. Wheat unhulled grains and flour grains may be classifications according to the degree to which the wheat is ground, unhulled grains may be coarsely ground nuruk, and flour grains may be finely ground nuruk. Flour grains may have a higher degree of grinding, that is, a smaller average particle size than unhulled grains. Unhulled grains may refer to particles that pass through a 10 to 20 mesh screen, and flour grains may refer to particles that pass through a 60 to 80 mesh screen. Mesh is a unit used to express the mesh size of stainless steel mesh, and may refer to the number of spaces between 1 inch (25.4 mm).

In step (a), the nuruk prepared according to embodiments of the present invention may be further mixed in an amount of 2 to 4 parts by weight with respect to 100 parts by weight of wheat, and the fermented beverage prepared using the nuruk prepared according to embodiments of the present invention may be further mixed in an amount of 3 to 5 parts by weight with respect to 100 parts by weight of wheat. In this case, the nuruk and fermented beverage to be further mixed may also be prepared by a method that excludes further mixing the nuruk or fermented beverage, and may also be prepared by a method that further includes the step.

Step (b) may be a permeation step in which the dough is left to stand for 30 minutes to 3 hours such that water is uniformly absorbed into the dough.

Step (c) may specifically be a step of weighing a certain amount of dough in a mold, sealing the dough with a beam, compressing the dough to remove air layers, and then cutting the dough into a hexahedral shape or molding the dough using a forming mold.

According to an embodiment, the method may further include a step (c-1) of inoculating the molded dough with a strain composition between steps (c) and (d).

The strain composition may be an additive that is supplemented to more effectively induce the fermentation of the nuruk of the present invention.

The strain composition may include one or more selected from the group consisting of Aspergillus oryzae, Lichtheimia corymbifera, Monascus pilosus, Galactomyces candidus, and Rhizopus japonicus.

Specifically, the strain composition may include Galactomyces candidus.

More specifically, the strain composition may include Lichtheimia corymbifera, Monascus pilosus, and Galactomyces candidus.

More specifically, the strain composition may include Lichtheimia corymbifera, Monascus pilosus, and Galactomyces candidus at a weight ratio of 2:1 to 2:1 to 2.

More specifically, the strain composition may include Lichtheimia corymbifera, Monascus pilosus, and Galactomyces candidus at a weight ratio of 2:1:1.

The strain composition may be inoculated in an amount of 0.01 to 0.09 parts by weight with respect to 100 parts by weight of the molded dough. When the strain composition is inoculated in an amount less than 0.01 parts by weight, the improving effect of nuruk fermentation may be insignificant. When the strain composition is inoculated in an amount more than 0.09 parts by weight, excessive saccharification may occur or the quality may deteriorate.

Aspergillus oryzae is an organism belonging to the Ascomycota nuruk mold genus, and reproduces mostly asexually. Round spermathecae form at the ends of straight hyphae twigs, and from these grow a large number of densely packed twigs that are elongated, bottle-shaped, spherical, or club-shaped, and spores form at the ends of these. Individual spores are roughly spherical, but as the spores grow one after another, they form long chains that diverge at the ends thereof. The spores have degrading enzymes such as amylase, maltase, invertase, cellulase, and inulinase, and act to break down starch of amylase among the enzymes into sugar.

Lichtheimia corymbifera is also known as Absidia corymbifera, and is a genus of zygomycota and mucor. The lower part of the sporangium axis is swollen, and thus has a structure called apophysis.

Monascus pilosus is also known as red yeast rice mold, and has traditionally been used to make red yeast rice. By biosynthesizing statin-based compounds such as monacolin K, mevinolin, and lovastatin, which inhibit cholesterol biosynthesis, Monascus pilosus has the effect of preventing arteriosclerosis, promoting bone formation, and the like.

Galactomyces candidus is a type of yeast strain, and may promote the conversion of profilaggrin, a precursor of natural moisturizing factor (NMF), into natural moisturizing factors, and is rich in polysaccharide components, and thus may prevent moisture evaporation from the skin surface. In addition, Galactomyces candidus is rich in various vitamins such as vitamins B1 and B2, pantothenic acid, B6, and folic acid, and various natural enzymes such as protease, a saccharification enzyme produced during the fermentation process, have antioxidant, anti-aging, whitening, and skin moisturizing effects by enhancing the production and absorption of active substances.

Rhizopus japonicus is a zygomycete with large and usually black sporangia. The columella is hemispherical, and the hyphae grow profusely and elongate. Rhizopus japonicus has a strong starch saccharogenic power, and although it is a small amount, it has the ability to ferment alcohol and decompose proteins, and it also has strong ability to produce organic acids such as lactic acid and fumaric acid.

According to an embodiment of the present invention, the strain composition may further include honey.

Honey may promote the growth and fermentation of the strains included in the nuruk and strain composition, and may improve the quality of the nuruk and the fermented beverage prepared from the nuruk, such as taste, aroma, and flavor.

Honey may be further included in an amount of 0.1 to 1 parts by weight with respect to 100 parts by weight of the strain composition (before including honey). When honey is included in an amount less than 0.1 parts by weight, the effect of promoting the growth and fermentation of the strains may be insignificant. When honey is included in an amount more than 1 part by weight, the quality may deteriorate due to components included in the honey.

Each strain included in the strain composition of the present invention may be obtained by a method including: inoculating a culture medium with a specific strain; culturing the inoculum at 20 to 25° C. for 48 hours; inoculating a new culture medium with the culture solution again to perform culture at 20 to 25° C. for 48 hours; and collecting a culture product by centrifugation.

As the culture medium, it is possible to use a culture medium which includes peptone, dextrose, yeast extract, and malt extract. Specifically, the culture medium may be a mixture of 1 to 10 parts by weight of peptone, 10 to 20 parts by weight of dextrose, 1 to 10 parts by weight of yeast extract, and 1 to 10 parts by weight of malt extract with 1,000 parts by weight of water. However, the embodiments of the present invention are not limited thereto.

The first fermenting temperature in step (d) may be 35 to 50° C., specifically 40 to 45° C., and the primary culture period may be 8 to 12 days, specifically 9 to 11 days.

In step (d), a sufficient amount of one or more of pine needle and straw may be laid under or around the molded dough, and then the dough may be cultured. Specifically, pine needles or straws may be used in an amount sufficient to support (under) or surround (around) the molded dough.

The second culture temperature in step (e) may be 30 to 45° C., specifically 35 to 40° C., and the secondary culture period may be 5 to 10 days, specifically 6 to 9 days. In this case, the second fermenting temperature may be a temperature lower than the first fermenting temperature. When the first or second fermenting temperature is maintained within a predetermined range rather than at a specific temperature, the upper limit of the second fermenting temperature may be lower than the lower limit of the first fermenting temperature.

In step (e), during the secondary culture, wheat flour grain powder may be sprinkled on the dough in an amount of 3 to 5 parts by weight with respect to 100 parts by weight of wheat, to allow for more effective culture.

Step (f) may be a step of drying the second cultured dough by lowering the temperature to 25 to 40° C., specifically 30 to 35° C.

The aging period of step (g) may be from 15 days to 3 months, specifically 1 to 2 months.

Nuruk prepared by the method described above may have a saccharogenic power that is improved by 10 to 80%, specifically 20 to 60%, compared to that of the comparative group. The nuruk of the comparative group may refer to nuruk prepared under conditions where the first and second fermenting temperatures are the same or the first fermenting temperature is lower than the second fermenting temperature.

A method for preparing a nuruk-fermented beverage according to an embodiment of the present invention includes: (a) washing rice or non-glutinous rice; (b) steaming and ripening the washed raw material; (c) cooling the steamed and ripened raw material; (d) mixing the cooled raw material with nuruk and water; (e) subjecting the mixture to a primary fermentation at a first temperature; (f) subjecting the primary fermented mixture to a secondary fermentation at a second temperature higher than the first temperature; and (g) filtering the secondary fermented mixture.

In step (a), the rice or non-glutinous rice may be washed 2 to 4 times.

The raw material washed in step (b) may be steamed and ripened using steam, the steam temperature may be 100 to 120° C., and the steaming and ripening time may be 40 to 70 minutes.

Step (c) may be a step of cooling the steamed and ripened raw material to 20 to 40° C. Specifically, as the cooling method, it is possible to select one or more of the methods of spraying water on the steamed and ripened raw material, naturally drying the steamed and ripened raw material, or applying wind to the steamed and ripened raw material. These methods may improve the flavor of the nuruk-fermented beverage of the present invention.

Step (d) may be a step of mixing 80 to 120 parts by weight of the cooled raw material, 5 to 20 parts by weight of nuruk, and 50 to 150 parts by weight of water. As the nuruk, it is possible to use one prepared by the method according to the embodiments of the present invention described above.

The first temperature in step (e) may be 20 to 30° C., and the primary fermentation period may be 1 to 4 weeks, specifically 2 to 3 weeks.

The second temperature in step (f) may be 25 to 35° C., and the secondary fermentation period may be 2 to 9 days, specifically 4 to 7 days. In this case, the second temperature may be maintained at a temperature higher than the first temperature. When the first temperature or the second temperature is maintained within a predetermined range rather than at a specific temperature, the lower limit of the second temperature may be higher than the upper limit of the first temperature.

In steps (e) and (f), the humidity may be maintained at 70 to 90%.

Step (g) may be a step of filtering or extracting the secondary fermented mixture (or raw liquid) using a filter with a mesh size of 100 to 140, specifically, 110 to 130. When the filtration step as described above is performed, lees and filtered fermented beverage may be obtained at a ratio of 10 to 20 parts by weight and 100 to 300 parts by weight, and the filtered fermented beverage may be a fermented wine with an alcohol content of about 15%.

The fermented food composition or health functional food composition according to an embodiment of the present invention may include nuruk prepared by the method or may be fermented with the nuruk, and the form thereof is not limited, and the content thereof is also not limited as long as the activity of the nuruk can be exhibited.

The health functional food of the present invention may contain various flavoring agents or natural carbohydrates, and the like as additional ingredients. The above-described natural carbohydrates are monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin or aspartame, and the like.

In addition to the aforementioned ingredients, the health functional food of the present invention may contain various nutrients, vitamins, electrolytes, flavors, colorants, pectic acids and salts thereof, alginic acid and salts thereof, protective colloid thickeners, pH adjusters, stabilizers, preservatives, glycerin, alcohols, carbonation agents used in carbonated beverages, and the like. These ingredients may be used either alone or in combination.

The nuruk-fermented cosmetic composition according to an embodiment of the present invention includes a nuruk-fermented product as an active ingredient.

The nuruk-fermented product may be obtained by suspending nuruk in water, then filtering the suspension with a filter, concentrating the filtered suspension under reduced pressure, and then filtering the concentrate with a filter again. Water may be used in an amount of 3- to 10-fold the weight of the nuruk, concentration under reduced pressure may be performed until the Brix is 5 to 10, and the size of the filter may be 1 to 10 km.

In addition, the nuruk-fermented product may be further subjected to a step of mixing nuruk with ethanol, concentrating the mixture under reduced pressure, and then filtering the concentrate with a filter. Ethanol may be used in an amount of 3- to 10-fold the weight of the nuruk, concentration under reduced pressure may be performed until the Brix is 5 to 10, and the filter may be a ultrafiltration (UF) with a KDa of 1 to 5.

The nuruk may be prepared by the method of the above-described present invention. A detailed description of the method for preparing nuruk will be omitted because it has been described above.

The nuruk-fermented product may include a fermented product of Galactomyces candidus.

The nuruk-fermented product may be included in the nuruk-fermented cosmetic composition in an amount of 1 to 10 wt %, specifically 3 to 7 wt %, and more specifically about 5 wt %.

The nuruk-fermented cosmetic composition may further include one or more selected from the group consisting of green tea extract, shea butter, tocopheryl acetate, vegetable oil, sodium hyaluronate, aloe vera leaf juice, acetyl glucosamine, beta-glucan, glutathione, and adenosine.

The nuruk-fermented cosmetic composition according to an embodiment of the present invention may be intended to be used for one or more uses selected from the group consisting of skin soothing, moisturizing, whitening, brightening, anti-wrinkle, antioxidant, and vitality restoration.

The nuruk-fermented cosmetic composition of the present invention may be formulated by a method typically used in the art.

Specifically, the cosmetic composition may be prepared in the form of a general emulsion formulation and solubilization formulation. For example, the cosmetic composition may be formulated into a lotion (toner) such as a softening lotion and a nourishing lotion; an emulsion such as a facial lotion and a body lotion; a cream such as BB cream, sun cream, nourishing cream, moisture cream, and eye cream; essence; a cosmetic ointment; spray; gel; pack; makeup base: a foundation such as a liquid, solid, or spray type; powder; a makeup remover such as cleansing cream, cleansing lotion, and cleansing oil; or a cleansing agent such as cleansing foam, soap, and body wash, and the like, but is not limited thereto.

The cosmetic composition may include a typically acceptable carrier, and may be obtained by appropriately blending, for example, an oil component, water, a surfactant, a moisturizer, a lower alcohol, a thickener, a chelating agent, a coloring matter, a preservative, a fragrance, and the like, but is not limited thereto.

The cosmetic composition may further include a carrier that is typically acceptable depending on the formulation. For example, when the cosmetic composition is formulated into a paste, cream or gel, animal oil, vegetable oil, wax, paraffin, starch, tragacanth, a cellulose derivative, polyethylene glycol, silicone, bentonite, silica, talc, zinc oxide or a mixture thereof may be used as a carrier component.

When the cosmetic composition is formulated into a powder or spray, lactose, talc, silica, aluminum hydroxide, calcium silicate, polyamide powder or a mixture thereof may be used as a carrier component, and in the case of a spray, a propellant such as a chlorofluorohydrocarbon, propane, butane or dimethyl ether may be further included.

When the cosmetic composition is formulated into a solution or emulsion, a solvent, a solubilizer, or an emulsifier may be used as a carrier component, for example, water, ethanol, isopropanol, ethyl carbonate, ethyl acetate, benzyl benzoate, propylene glycol, and 1,3-butyl glycol oil may be used, and in particular, cottonseed oil, peanut oil, corn germ oil, olive oil, castor oil and sesame oil, glycerol aliphatic esters, and fatty acid esters of polyethylene glycol or sorbitan may be used.

When the cosmetic composition is formulated into a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier component.

When the cosmetic composition is formulated into soap, an alkali metal salt of a fatty acid, a fatty acid hemiester salt, a fatty acid protein hydrolysate, isethionate, a lanoline derivative, an aliphatic alcohol, vegetable oil, glycerol, sugar, and the like may be used as a carrier component.

The cosmetic composition may further contain an auxiliary typically used in the cosmetological or dermatological fields, such as a lipid material, an organic solvent, a solubilizing agent, a thickening agent, a gelling agent, an emollient, an antioxidant, a suspending agent, a stabilizer, a foaming agent, a fragrance ingredient, a surfactant, water, an ionic or nonionic emulsifying agent, a filler, a metal ion chelating agent, a chelating agent, a preservative, a blocking agent, a wetting agent, an essence oil, a dye, a pigment, a hydrophilic or lipophilic active agent typically used in the art, or any other components typically used in a cosmetic, according to the quality or function of a final product.

The cosmetic composition may further include a peptide that is typically used in dermatological compositions for improving skin. For example, the cosmetic composition may further include one or more selected from the group consisting of SH-oligopeptide-1, SH-oligopeptide-2, SH-polypeptide-1, SH-polypeptide-16, and SH-polypeptide-3, but is not limited thereto.

Hereinafter, the present invention will be described through Preparation Examples and Experimental Examples, but it is obvious that the effects of the present invention are not limited by the following Experimental Examples.

### Preparation Example 1: Preparation of Nuruk

### Example 1-1

Wheat kernels that were separated from stones using a winnowing machine were ground coarsely such that no raw grains remained, thereby obtaining wheat unhulled grains. After 120 kg of wheat was mixed with 25 L of splash water and kneaded for 30 minutes, the resulting mixture was left to stand for 1 hour such that the water was absorbed evenly into the dough. A certain amount of dough was weighed in a forming mold, sealed with a beam, and then compressed to remove air layers, and then molded into a diameter of 20 cm and a thickness of 3 cm in size. The dough was cultured at 40° C. for 10 days as a primary culture, and then at 35° C. for 7 days as a secondary culture. The dough was gradually dried by lowering the fermenting temperature to 30° C., and then aged for one month.

### Example 1-2

Nuruk was prepared in the same manner as in Example 1-1, except that wheat consisting of 80 wt % of unhulled grains and 20 wt % of flour grains was used as the wheat.

### Example 1-3

Nuruk was prepared in the same manner as in Example 1-1, except that 3.6 kg of the nuruk in Example 1-1 was further added before kneading.

### Example 1-4

Nuruk was prepared in the same manner as in Example 1-1, except that the dough was cultured by laying pine needles and straws under the dough during the primary culture.

### Example 1-5

Nuruk was prepared in the same manner as in Example 1-1, except that 4.8 kg of the nuruk-fermented beverage of Example 2-1 in the following Preparation Example 2 was further added before kneading.

### Example 1-6

Nuruk was prepared in the same manner as in Example 1-1, except that wheat flour grain powder was sprinkled on the dough during the secondary culture.

### Comparative Example 1-1

Nuruk was prepared in the same manner as in Example 1-1, except that the dough was cultured only at 40° C. for 20 days.

### Comparative Example 1-2

Nuruk was prepared in the same manner as in Example 1-1, except that the dough was cultured at 35° C. for 7 days as a primary culture, and then at 40° C. for 10 days as a secondary culture.

### Preparation Example 2: Preparation of Nuruk-Fermented Beverage

### Example 2-1

Washed rice was steamed and ripened using steam at 110° C. for 50 minutes. The steamed and ripened raw material was cooled to 30° C., and 100 kg of the cooled raw material was mixed with 10 kg of the nuruk prepared in Example 1-1 and 100 L of water. The mixture was subjected to primary fermentation at 25° C. and 70% humidity for 2 weeks, and then secondary fermentation at 30° C. and 70% humidity for 4 days. The fermented mixture was extracted using a filter with a mesh size of 120.

### Example 2-2

A nuruk-fermented beverage was prepared in the same manner as in Example 2-1, except that the nuruk prepared in Example 1-2 was used as the nuruk.

### Example 2-3

A nuruk-fermented beverage was prepared in the same manner as in Example 2-1, except that the nuruk prepared in Example 1-3 was used as the nuruk.

### Example 2-4

A nuruk-fermented beverage was prepared in the same manner as in Example 2-1, except that the nuruk prepared in Example 1-4 was used as the nuruk.

### Example 2-5

A nuruk-fermented beverage was prepared in the same manner as in Example 2-1, except that the nuruk prepared in Example 1-5 was used as the nuruk.

### Example 2-6

A nuruk-fermented beverage was prepared in the same manner as in Example 2-1, except that the nuruk prepared in Example 1-6 was used as the nuruk.

### Example 2-7

A nuruk-fermented beverage was prepared in the same manner as in Example 2-1, except that the steamed and ripened raw material was sprayed with water until it was cooled to 30° C.

### Comparative Example 2-1

A nuruk-fermented beverage was prepared in the same manner as in Example 2-1, except that the nuruk prepared in Comparative Example 1-1 was used as the nuruk.

### Comparative Example 2-2

A nuruk-fermented beverage was prepared in the same manner as in Example 2-1, except that the nuruk prepared in Comparative Example 1-2 was used as the nuruk.

### Comparative Example 2-3

A nuruk-fermented beverage was prepared in the same manner as in Example 2-1, except that the mixture was fermented only at 25° C. for 3 weeks.

### Comparative Example 2-4

A nuruk-fermented beverage was prepared in the same manner as in Example 2-1, except that the mixture was fermented only at 30° C. for 3 weeks.

### Comparative Example 2-5

A nuruk-fermented beverage was prepared in the same manner as in Example 2-1, except that the mixture was subjected to primary fermentation at 30° C. for 2 weeks, and then to secondary fermentation at 25° C. for 4 days.

### Experimental Example 1 Sensory Evaluation of Nuruk-Fermented Beverage

In order to evaluate the preference for nuruk-fermented beverages using the nuruk prepared by the method of the present invention, a sensory evaluation was performed on the nuruk-fermented beverages of the Examples and Comparative Examples of Preparation Example 2.

The sensory evaluation was performed by selecting 20 adult men and women in their 20s to 50s, with 5 people in each group evenly divided by age and sex, and the nuruk-fermented beverages of the Examples and Comparative Examples of Preparation Example 2 were provided to the participants to evaluate the taste, smell, smoothness on the throat, and overall preference. The ratings were made using a 10-point rating system, with higher scores being given for greater preference. After taking a sip of each nuruk-fermented beverage, the participants were asked to evaluate each item, rinse their mouths with water, and then evaluate the items by repeating the process of taking another fermented beverage after 5 minutes. The evaluation results are shown in the following Table 1.

TABLE 1

| | | | Smoothness | Comprehensive |
|---|---|---|---|---|
| Classification | Taste | Aroma | on the throat | evaluation |
| Example 2-1 | 8.5 | 8.3 | 8.8 | 8.5 |
| Example 2-2 | 8.9 | 9.0 | 9.3 | 9.0 |
| Example 2-3 | 8.6 | 9.2 | 8.9 | 8.9 |
| Example 2-4 | 8.8 | 9.4 | 8.8 | 9.0 |
| Example 2-5 | 9.5 | 9.3 | 9.0 | 9.2 |
| Example 2-6 | 8.9 | 8.7 | 9.0 | 8.8 |
| Example 2-7 | 8.4 | 8.6 | 9.2 | 8.7 |
| Comparative | 7.6 | 8.0 | 7.7 | 7.7 |
| Example 2-1 | | | | |
| Comparative | 8.1 | 8.5 | 7.8 | 8.0 |
| Example 2-2 | | | | |
| Comparative | 7.5 | 7.6 | 7.8 | 7.6 |
| Example 2-3 | | | | |
| Comparative | 7.3 | 7.5 | 7.5 | 7.4 |
| Example 2-4 | | | | |
| Comparative | 8.3 | 8.1 | 7.8 | 8.1 |
| Example 2-5 | | | | |

As shown in Table 1 above, it can be seen that the nuruk-fermented beverages of the Examples had a higher overall preference than the nuruk-fermented beverages of the Comparative Examples. In particular, Examples 2-2 to 2-5 showed higher preference such as taste and aroma than Example 2-1.

### Experimental Example 2: Characteristics of Nuruk or Nuruk-Fermented Beverages

The saccharogenic power (SP) of the rice nuruk prepared by the method of the present invention was analyzed according to the liquor analysis regulation.

Specifically, the amount of reducing sugar (mg) produced when 1 g of sample nuruk was reacted with a substrate which is a 2% soluble starch solution at 55° C. for 1 hour was expressed as SP (mg/hour), and calculated by multiplying the saccharogenic rate in a range where the saccharogenic rate of the substrate was 15% by the dilution factor.

TABLE 2

| Classification | Saccharogenic power (SP) |
|---|---|
| Example 1-1 | 705.56 |
| Example 1-3 | 710.23 |
| Example 1-5 | 790.96 |
| Comparative Example 1-1 | 563.28 |
| Comparative Example 1-2 | 348.84 |

As shown in Table 2 above, it can be seen that the nuruk in each of the Examples had a better overall saccharogenic power than the nuruk in each of the Comparative Examples.

### Preparation Example 3: Preparation of Nuruk (2)

### Example 3-1

Nuruk was prepared in the same manner as in Example 1-1, except that the molded dough was inoculated with a strain composition including Galactomyces candidus in an amount of 0.02 parts by weight with respect to 100 parts by weight of the dough.

### Example 3-2

Nuruk was prepared in the same manner as in Example 1-1, except that the molded dough was inoculated with a strain composition including Lichtheimia corymbifera, Monascus pilosus, and Galactomyces candidus at a weight ratio of 2:1:1 in an amount of 0.02 parts by weight with respect to 100 parts by weight of the dough.

### Example 3-3

Nuruk was prepared in the same manner as in Example 3-1, except that honey was added to the strain composition in an amount of 0.5 parts by weight with respect to 100 parts by weight of the strain composition.

### Comparative Example 3-1

Nuruk was prepared in the same manner as in Example 3-1, except that the strain composition included Aspergillus oryzae, Monascus pilosus, and Galactomyces candidus at a weight ratio of 1:1:1.

### Comparative Example 3-2

Nuruk was prepared in the same manner as in Example 3-1, except that the strain composition included Lichtheimia corymbifera, Monascus pilosus, and Galactomyces candidus at a weight ratio of 1:2:2.

### Preparation Example 4: Preparation of Nuruk-Fermented Beverage (2)

### Example 4-1

A nuruk-fermented beverage was prepared in the same manner as in Example 2-1, except that the nuruk prepared in Example 3-1 was used as the nuruk.

### Example 4-2

A nuruk-fermented beverage was prepared in the same manner as in Example 4-1, except that the nuruk prepared in Example 3-2 was used as the nuruk.

### Example 4-3

A nuruk-fermented beverage was prepared in the same manner as in Example 4-1, except that the nuruk prepared in Example 3-3 was used as the nuruk.

### Comparative Example 4-1

A nuruk-fermented beverage was prepared in the same manner as in Example 4-1, except that the nuruk prepared in Comparative Example 3-1 was used as the nuruk.

### Comparative Example 4-2

A nuruk-fermented beverage was prepared in the same manner as in Example 4-1, except that the nuruk prepared in Comparative Example 3-2 was used as the nuruk.

### Preparation Example 5: Preparation of Nuruk-Fermented Carbonated Beverage

### Example 5-1

After 0.01 parts by weight of beer yeast was added to the mixture prepared in Example 2-1, the mixture was fermented in a sealed container at 15° C. for 20 hours, and then the container was rapidly cooled.

### Example 5-2

After 0.1 parts by weight of carbon dioxide gas was injected into the mixture prepared in Example 2-1, the container was sealed, and then cooled.

### Example 5-3

After 0.01 parts by weight of beer yeast and 0.1 parts by weight of carbon dioxide gas were added to the mixture prepared in Example 2-1, the mixture was fermented in a sealed container at 15° C. for 20 hours, and then the container was rapidly cooled.

### Experimental Example 3: Sensory Evaluation of Nuruk-Fermented Beverage (2)

In order to evaluate the preference for nuruk-fermented beverages using the nuruk prepared by the method of the present invention, a sensory evaluation was performed on the nuruk-fermented beverages of the Examples and Comparative Examples of Preparation Example 4 and 5.

The sensory evaluation was performed by selecting 20 adult men and women in their 20s to 50s, with 5 people in each group evenly divided by age and sex, and the nuruk-fermented beverages of the Examples and Comparative Examples of Preparation Example 4 were provided to the participants to evaluate the taste, smell, smoothness on the throat, and overall preference. The ratings were made using a 10-point rating system, with higher scores being given for greater preference. After taking a sip of each nuruk-fermented beverage, the participants were asked to evaluate each item, rinse their mouths with water, and then evaluate the items by repeating the process of taking another fermented beverage after 5 minutes. The evaluation results are shown in the following Table 3.

TABLE 3

| | | | Smoothness | Comprehensive |
|---|---|---|---|---|
| Classification | Taste | Aroma | on the throat | evaluation |
| Examples 4-1 | 8.6 | 8.8 | 8.7 | 8.7 |
| Examples 4-2 | 8.3 | 8.8 | 9.0 | 8.7 |
| Examples 4-3 | 8.4 | 9.2 | 8.8 | 8.8 |
| Examples 5-1 | 8.2 | 8.7 | 9.3 | 8.7 |
| Examples 5-2 | 8.9 | 8.9 | 8.7 | 8.8 |
| Examples 5-3 | 8.8 | 9.0 | 9.1 | 9.0 |
| Comparative | 8.4 | 8.6 | 8.8 | 8.6 |
| Example 4-1 | | | | |
| Comparative | 8.5 | 7.9 | 8.8 | 8.4 |
| Example 4-2 | | | | |

As shown in Table 3 above, it can be seen that the nuruk-fermented beverages of the Examples had a higher overall preference than the nuruk-fermented beverages of the Comparative Examples.

### Experimental Example 4: Characteristics of Nuruk or Nuruk-Fermented Beverages (2)

The saccharogenic power (SP) of the rice nuruk prepared by the method of the present invention was analyzed according to the liquor analysis regulation.

Specifically, the amount of reducing sugar (mg) produced when 1 g of sample nuruk was reacted with a substrate which is a 2% soluble starch solution at 55° C. for 1 hour was expressed as SP (mg/hour), and calculated by multiplying the saccharogenic rate in a range where the saccharogenic rate of the substrate was 15% by the dilution factor.

TABLE 4

| Classification | Saccharogenic power (SP) |
|---|---|
| Example 3-1 | 695.66 |
| Example 3-2 | 717.84 |
| Example 3-3 | 709.56 |
| Comparative Example 3-1 | 695.20 |
| Comparative Example 3-2 | 690.32 |

As shown in Table 4 above, it can be seen that the nuruk in each of the Examples had a better overall saccharogenic power than the nuruk in each of the Comparative Examples.

### Preparation Example 6: Preparation of Nuruk-Fermented Cosmetic Composition

### Example 6-1

The nuruk prepared in Example 1-1 was suspended in 3 times its weight of purified water and filtered through a 5 m filter. The filtrate was concentrated under reduced pressure to 10 Brix and then filtered through a 0.4 m filter to prepare a nuruk-fermented product. The nuruk-fermented product was mixed with cosmetic excipients such as purified water, an emulsifier, a lubricant, a stabilizer, and a preservative to prepare a cosmetic composition in the form of a cream formulation.

### Example 6-2

A nuruk-fermented cosmetic composition was prepared in the same manner as in Example 6-1, except that the nuruk prepared in Example 2-1 was used as the nuruk.

### Comparative Example 6-1

A nuruk-fermented cosmetic composition was prepared in the same manner as in Example 6-1, except that the nuruk prepared in Comparative Example 1-1 was used as the nuruk.

### Comparative Example 6-2

A nuruk-fermented cosmetic composition was prepared in the same manner as in Example 6-1, except that rice nuruk prepared by a publicly-known method was used as the nuruk.

### Experimental Example 5: Efficacy Evaluation of Nuruk-Fermented Cosmetic Composition

### 1) Evaluation of the Effect of Increasing the Amount of a Moisturizing Factor Filaggrin (FLG) Produced

HaCaT cells were aliquoted in a 24-well plate at 1.0×10⁵ cells/well and cultured under the conditions of 37° C. and 5% CO₂ for 18 hours.

After the medium for the cultured cells was replaced with serum-free DMEM medium, the cells were treated with each of the nuruk-fermented cosmetic compositions of the Examples and Comparative Examples of Preparation Example 6 at a concentration of 0.5 vol %, and cultured for 24 hours.

Thereafter, the cells of each experimental group were lysed, and then a protein was taken to measure the amount of filaggrin produced in the culture solution extract. The control was treated with retinoic acid at a concentration of 10 μM.

The amount of filaggrin produced was measured using a Filaggrin ELISA kit (Cusabio Biotechnology Co., Ltd.) according to the method provided by the manufacturer. The results of measuring the amount of filaggrin produced are shown in the following Table 5.

TABLE 5

| Classification | Amount of FLG produced (%) |
|---|---|
| Example 6-1 | 63.78 ± 0.11 |
| Example 6-2 | 65.34 ± 0.23 |
| Comparative Example 6-1 | 64.12 ± 0.14 |
| Comparative Example 6-2 | 37.23 ± 0.43 |
| Control | 100 ± 0.05 |

As shown in Table 5 above, it can be seen that the nuruk-fermented cosmetic compositions of the Examples had a better overall moisturizing effect than the nuruk-fermented cosmetic compositions of the Comparative Examples.

### 2) Antioxidant Effect Measurement

An antioxidant effect was measured through a free radical scavenging test using the absorbance of DPPH.

The nuruk-fermented products of the Examples and Comparative Examples of Preparation Example 6 were each diluted with methanol to a concentration of 0.5% to prepare diluted solutions of the nuruk-fermented products. A sample was prepared by mixing 1 ml of the nuruk-fermented product diluted solution with 1 ml of a 0.1 mM 2,2-diphenyl-1-picryl-hydrazyl radical (DPPH, Sigma) solution. After the prepared sample was left to stand at room temperature for 15 minutes, absorbance was measured at a wavelength of 517 nm using a microplate reader.

As a negative control, a solution obtained by mixing 1 ml of DPPH with 1 ml of methanol was used, and as a blank sample for obtaining a color correction value for each of the sample and the control, a solution obtained by mixing 1 ml of methanol with 1 ml of the sample was used. Furthermore, a positive control in the present experimental example was prepared by mixing 1 ml of an ascorbic acid (10 ppm) solution diluted in methanol with 1 ml of DPPH.

TABLE 6

| Classification | DPPF radical scavenging activity (%) |
|---|---|
| Example 6-1 | 69.11 ± 0.17 |
| Example 6-2 | 72.74 ± 0.21 |
| Comparative Example 6-1 | 60.45 ± 0.09 |
| Comparative Example 6-2 | 63.56 ± 0.12 |
| Ascorbic acid | 86.86 ± 0.21 |
| Untreated group | 0 |

As shown in Table 6 above, it can be seen that the nuruk-fermented cosmetic compositions of the Examples had better overall antioxidant efficacy than the nuruk-fermented cosmetic compositions of the Comparative Examples.

The nuruk for fermentation prepared according to the embodiments of the present invention can prepare a nuruk-fermented beverage with excellent preference such as taste and aroma, and has excellent saccharogenic power.

The effects according to the embodiments of the present invention are not limited to the contents exemplified above, and more various effects are included in the present specification.

Although the present invention has been described above with reference to embodiments thereof, these are merely examples and do not limit the present invention. Those skilled in the art will appreciate that various modifications and applications not exemplified above are possible without departing from the essential characteristics of the embodiments of the present invention. For example, each component specifically shown in the embodiments of the present invention can be modified and implemented. In addition, the differences related to such modifications and applications should be interpreted as being included in the scope of the present invention defined in the appended claims.

What is claimed is:

1. A method for preparing nuruk-fermented carbonated beverage, the method comprising:
(a) washing rice or non-glutinous rice;
(b) steaming and ripening the washed raw material;
(c) cooling the steamed and ripened raw material;
(d) mixing the cooled raw material with nuruk and water;
(e) subjecting the mixture to a primary fermentation at a first temperature;
(f) subjecting the primary fermented mixture to a secondary fermentation at a second temperature higher than the first temperature;
(g) filtering the secondary fermented mixture;
(h-1) adding yeast to the filtered mixture and then subjecting the resulting mixture to a third fermentation in a sealed container; and
(i) rapidly cooling the container.

2. The method of claim 1, wherein the nuruk is obtained by a method comprising:
(a) kneading a mixture of wheat and water;
(b) leaving the resulting dough to stand for a predetermined time;
(c) molding the dough into a predetermined shape;
(d) a primary culture step of culturing the molded dough at a first fermenting temperature;
(e) a secondary culture step of culturing the primary cultured dough at a second fermenting temperature lower than the first fermenting temperature;
(f) drying the secondary cultured dough by lowering a fermenting temperature of the secondary cultured dough; and
(g) aging the dried dough for a predetermined time.

3. The method of claim 2, wherein the wheat in step (a) comprises 70 to 90 parts by weight of wheat unhulled grains and 10 to 30 parts by weight of wheat flour grains.

4. The method of claim 2, wherein in step (a), nuruk prepared by the same method of claim 2 is further mixed in an amount of 2 to 4 parts by weight with respect to 100 parts by weight of the wheat.

5. The method of claim 2, wherein in step (d), one or more of pine needle and straw are laid under or around the molded dough, and then the dough is cultured.

6. The method of claim 2, wherein the first fermenting temperature is 35 to 50° C. and the primary culture period is 8 to 12 days, and the second fermenting temperature is 30 to 45° C. and the secondary culture period is 5 to 10 days.

7. The method of claim 2, further comprising sprinkling wheat flour grains on the dough in an amount of 3 to 5 parts by weight with respect to 100 parts by weight of the wheat during the secondary culture in step (e).

8. The method of claim 2, further comprising (c-1) inoculating the molded dough with a strain composition between steps (c) and (d).

9. The method of claim 8, wherein the strain composition comprises one or more selected from the group consisting of Aspergillus oryzae, Lichtheimia corymbifera, Monascus pilosus, Galactomyces candidus, and Rhizopus japonicus.

10. The method of claim 9, wherein the strain composition comprises Galactomyces candidus.

11. The method of claim 10, wherein the strain composition comprises Lichtheimia corymbifera, Monascus pilosus, and Galactomyces candidus.

12. The method of claim 11, wherein the strain composition comprises Lichtheimia corymbifera, Monascus pilosus, and Galactomyces candidus at a weight ratio of 2:1 to 2:1 to 2.

13. The method of claim 8, wherein each strain comprised in the strain composition is obtained by a method comprising:
inoculating a culture medium with a specific strain;
culturing an inoculum at 20 to 25° C. for 48 hours;
inoculating a new culture medium with the culture solution again to perform culture at 20 to 25° C. for 48 hours; and
collecting a culture product by centrifugation.

14. The method of claim 8, wherein the strain composition further comprises honey.

15. The method of claim 1, wherein step (d) is a step of mixing 80 to 120 parts by weight of the cooled raw material, 5 to 20 parts by weight of nuruk, and 50 to 150 parts by weight of water.

16. The method of claim 1, wherein the second temperature is higher than the first temperature.

17. The method of claim 1, wherein the first temperature in step (e) is 20 to 30° C., and the primary fermentation period is 1 to 4 weeks, wherein the second temperature in step (f) is 25 to 35° C., and the secondary fermentation period is 2 to 9 days.

18. The method of claim 1, wherein the yeast is beer yeast.

19. A method for preparing nuruk-fermented carbonated beverage, the method comprising:
(a) washing rice or non-glutinous rice;
(b) steaming and ripening the washed raw material;
(c) cooling the steamed and ripened raw material;
(d) mixing the cooled raw material with nuruk and water;
(e) subjecting the mixture to a primary fermentation at a first temperature;
(f) subjecting the primary fermented mixture to a secondary fermentation at a second temperature higher than the first temperature;
(g) filtering the secondary fermented mixture; and
(h-2) injecting carbon dioxide gas into the filtered mixture and then sealing the mixture.

20. A method for preparing nuruk-fermented carbonated beverage, the method comprising:
(a) washing rice or non-glutinous rice;
(b) steaming and ripening the washed raw material;
(c) cooling the steamed and ripened raw material;
(d) mixing the cooled raw material with nuruk and water;
(e) subjecting the mixture to a primary fermentation at a first temperature;
(f) subjecting the primary fermented mixture to a secondary fermentation at a second temperature higher than the first temperature;
(g) filtering the secondary fermented mixture;
(h-3) injecting yeast and carbon dioxide gas into the filtered mixture and then subjecting the resulting mixture to a third fermentation in a sealed container; and
(i) rapidly cooling the container.
